(19) United States
(12) Patent Application Publication
(10) Pub. No.: US 2014/0081177 A1
(43) Pub. Date: Mar. 20, 2014

(54) POSTUROGRAPHIC SYSTEM USING A BALANCE BOARD

(71) Applicants: Enrique Soto Eguibar, Col. San Manuel (MX); María Del Rosario Guadalupe Vega Y Saénz De Miera, Col. San Manuel (MX); Eduardo Salinas Márquez, Col.San Manuel (MX); Paulina Robles Hortega, Col. San Manuel (MX)

(72) Inventors: Enrique Soto Eguibar, Col. San Manuel (MX); María Del Rosario Guadalupe Vega Y Saénz De Miera, Col. San Manuel (MX); Eduardo Salinas Márquez, Col.San Manuel (MX); Paulina Robles Hortega, Col. San Manuel (MX)

(73) Assignee: BENEMERITA UNIVERSIDAD AUTONOMA DE PUEBLA, Puebla (MX)

(21) Appl. No.: 13/618,477

(22) Filed: Sep. 14, 2012

(51) Int. Cl. A61B 5/103

(52) U.S. Cl. CPC A61B 5/1036
USPC 600/595

(57) ABSTRACT

A stabilometric system is provided that uses a balance platform to detect problems in the vestibular system via data capture, data visualization and mathematical analysis of data, the system having means for data capture that obtain customized records and store data resulting from the readings of the sensors of the balance platform, means for displaying the data obtained using stabilometric tests on a screen that is controlled by a computer, and means for processing the data obtained from the measurements.

### BACKGROUND

1. Technical Field of the Invention

The present invention relates to a stabilometric system, for detecting problems in the vestibular system and the influences that certain drugs or medicines may have in the balance of people.

2. General Background of the Invention

The study of the ability of the subjects to maintain an upright position, is known as stabilometry and provides information about the function of this set of systems related with the maintenance of the balance. Since stabilometry is a non-invasive and simple test, is increasingly applied to study the effects of various environmental elements as well as drugs that may cause damage or effects to the central nervous system, or the alterations that may be caused in posture and balance (i.e., affecting any of the systems responsible for maintaining it). The functioning is, broadly speaking, the obtaining of the pressure shifts exercised by the feet, using pressure sensors located on the vertices of triangular or square platforms (Nishiwaki of al. 1999).

According to studies carried out by the Japanese Society for Equilibrium Research (JSER 1983) certain procedures for the use of stabilometry have been standardized, such as:

1) During the test, the legs may not be separated.
2) The upper limbs should be aligned at the sides of the torso.
3) The individuals examined may be placed in a naturally straight position.

However, there is a problem, because there is no standard for the instructions of how the examiner should indicate the subject or examined individual their position. In 1999 (Nishiwaki et al. 1999) a study on how to give instructions to the examined individuals when standing on the platform in a stabilometric test was carried out, and it was concluded that when explaining in a different way the instructions, the subjects had changes in their oscillation (there was a greater shift in cm with one instruction than with the other).

Typically, the injuries of the vestibular system (inner ear), are accompanied by loss of balance, which is why stabilometry offers information that can contribute to the diagnosis in subjects suspected of vestibular damage (Halmagyi et al. 1996).

The definition of symptoms and diseases is a fundamental prerequisite for disciplines that depend largely on diagnoses based on symptoms, and where often there is no available independent diagnosis standard.

Symptoms Associated with the Vestibular System

There are different definitions for the symptoms related with diseases in the vestibular system, but according to Brisdorff, the International Classification of Vestibular Disorders I (ICVD-I) defines the following conditions:

1. Vertigo.—The feeling of proper motion when nothing actually occurs, or else, the feeling of proper motion distorted during a normal movement of the head. There are several types of vertigo which are: a) Spontaneous vertigo; b) Induced vertigo; c) Positional vertigo; d) Head motion vertigo; e) Visually-induced vertigo; f) Aurally-induced vertigo; g) Vertigo induced by the Valsalva manoeuvre; h) Ortho-static vertigo and i) Vertigo caused by other reasons.
2. Dizziness.—It is the disturbed or damaged sense of spatial orientation, without a false or distorted sense of movement. There are several types of dizziness which are: a) Spontaneous dizziness; b) Induced dizziness; c) Positional dizziness; d) Head motion dizziness; e) Visually-induced dizziness; f) Aurally-induced dizziness; g) Dizziness induced by the Valsalva manoeuvre; h) Ortho-static Dizziness and i) Dizziness caused by other reasons.
3. Vestibular-visual symptoms.—These are visual symptoms that usually result from a vestibular pathology, or from the interaction between these two systems. There are several types of Vestibular-visual symptoms which are: a) Vertigo (external); b) Oscillopsia; c) Visual lag; d) Visual tilt and e) Movement-induced blur.
4. Postural symptoms.—These are balance symptoms related with the stability that occur only while the person is upright (sitting, standing or walking). There are several types of postural symptoms which are: a) Unsteadiness; b) Directional pulsion; c) Balance related near fall and d) Balance related fall.

In 1995 a comparison of the two types of posturography: dynamic and static (Di Fabio 1995) was carried out. Various previously carried out studies were collected and it was concluded that static posturography is more sensitive for the detection of vestibular peripheral deficit than dynamic posturography.

It has been determined that the rehabilitation exercises for the vestibular system may be suitable for each subject, depending on the previously retrieved diagnosis.

It has been observed that on firm and flat surfaces, the somatosensory or proprioceptive information is the most important in providing information to control the position, while on unstable or moving surfaces, the vestibular system is the one that provides more useful information to control the position (Mergner et al. 1997).

The use of the computerized dynamic posturography (CDP) has been studied for the stage of the diseases of the vestibular system, in particular Meniere disease. The use of dynamic posturography in the diagnosis of subjects with balance disorders, not only allows the quantification of the capacity of the subject for keeping their centre of gravity stable, but also the analysis of the degree in which the subject can use different types of sensory information (Soto et al. 2004).

In 2006 was carried out a study in which it was determined that the lower frequencies of oscillation of the body in the vertical position are linked with the visual control, the medium-low frequencies are linked with the vestibular system, the medium-high frequencies with the proprioceptive system and finally the highest frequencies indicated an abrupt change in the posture as well as damage to the nervous system. Based on this information, in our system it may be used the analysis with the Fast Fourier Transform to detect alterations in these frequency bands, focusing the attention on the band associated with the vestibular system (Avni et al. 2006).

The computerized dynamic posturography (CDP) has proven to be a cheap technique and useful for the characterization and monitoring of subjects with balance problems. CDP obtains important information about the functional state of the balance and the ability of the subject to take advantage of the information received by the vestibular, proprioceptive and visual systems (Stewart et al. 1999).

The systems and apparatus for the detection and diagnosis of problems associated with the vestibular system, are scarce and expensive, which is why an economic solution is required that serves as an alternative to these.

The United States patent application US-2011/0218077 A1 (FERNANDEZ), describes a device for measuring strength by extending the capabilities of a weight and balance detection platform, such as the Wii balance board (Wii Balance Board, manufactured by Nintendo). The apparatus has a base unit configured to keep secure a weight and balance detection platform and it has an anchorage point to which is attached the mechanism of resistance. A user placed on the weight and balance detection platform can exert a force on the mechanism of resistance that may be detected by the weight and balance detection platform together with any apparent shift in its balance centre caused by the force. These measurements are wirelessly transmitted to a computer and used to integrate the efforts of the user in a game or an exercise routine. The system of measurement of the effort can include anchor extensions that serve both as anchorage points for the mechanism of resistance and as legs, to provide additional stability.

The United States patent application US-2010/0228144 A1 (LABAT), describes an invention related to eye stimulation and posturography equipment, characterized in that they comprise, in combination: a support that can be fixed in a removable manner to the head of the subject and include at least one eye visibility device to be placed in front of an eye of the subject, each visibility device comprising an exhibition display and a hollow body, in which the screen is placed, and being designed to be placed in front of only one eye of the subject and to minimize the visual reference marks for the subject that are not those that appear on the display, means for significantly detecting reactions of the body in the subject, which are capable of delivering measurement signals representative of significant reactions of the body, means for the acquisition and recording of measurement signals delivered by the means of detection, means for synchronizing the transmitted image signals and the measurement signals received, as well as being able to correlate these two types of signals.

The international patent application WO-2007/0135462 A1 (SPEARS), describes a system and method for monitoring the balance in a person, for example when carrying out an assessment of posturography subsequently to a stroke, in which a unit with light emitting device on a given spatial arrangement is connected to the person. A system for monitoring the balance of a person, the system comprises: i) a bearing unit that contains at least one indication, or several indications with a default spatial configuration in the unit, and ii) an image capturing device, wherein the capture unit or the unit with indications can be joined to the person such that the unit or device is located in the centre of balance of a subject, the system is configured to measure the movement of the unit, and it is also configured to record the movement of at least one indication with reference to the centre of balance of the subject to obtain an objective measurement of the balance.

The United States patent application US-2008/0228110 A1 (NECIP), describes a device for balance training and evaluation of dynamic balance by means of the measurement of the ability of a subject to react to disturbances. A universal joint assembly is transferred to the base of a supporting surface while a top surface, wherein there is a subject, is fixed against the transfer. The universal joint allows the top surface to rotate around at least one and preferably multiple axes and the subject may have to control the balance following the transfer of the universal joint. All the components are placed on a one-piece platform assembly. A virtual environment by means of the devices of created images can be used to create a realistic sense of general posture movement and instability, or displacement of the supporting surface.

The previous systems constitute various expensive devices and away from their potential use in the study of people with posture alterations. Therefore, there is a need in the state of the art of a low-cost stabilometry system, based on the Wii balance board to detect problems in the vestibular system.

### SUMMARY OF THE INVENTION

An objective of the present invention consists of the obtaining and viewing of data relating to the ability of the user to maintain the balance and the monitoring of the balance of a person, as well as the creation of an individual record in which all the data from the tests performed to said person are stored.

Another objective of the invention consists of the processing of the data obtained, to determine the frequency of oscillations of the subject, that leads to determine if there are problems in the inner ear of a person.

Another objective of the invention is the implementation of corrective tests, with the purpose of helping the people to improve their stability, and training them to compensate for the problems that may have in case of having injuries in their vestibular system.

The above objectives are achieved through a stabilometric system using a balance platform to detect problems in the vestibular system characterized in that it comprises the steps of: i) data capture; ii) data visualization; iii) mathematical analysis of data. Said steps include: means for data capture consisting of the obtaining, customized registration and storage of the results of the readings of the sensors of the balance platform; presentation of the data obtained through the stabilometric tests on a display that is controlled by a computer; and means for processing the data obtained from the measurements.

### BRIEF DESCRIPTION OF THE DRAWINGS

The present invention will be completely understood through the detailed description given herein below and the attached schemes, which are given by way of illustration and example only and therefore are not limiting with respect to the aspects of the present invention, wherein:

FIG. 1 illustrates a flowchart corresponding to a first step, according to various aspects of the current invention.

FIG. 2 illustrates the user interface of the first step, according to various aspects of the current invention.

FIG. 3 illustrates a flowchart corresponding to a second step, according to various aspects of the current invention.

FIG. 4 illustrates an orientation scheme, which includes the position and the angle of sensitivity of the Wii board.

FIG. 5 illustrates the interface of the second step.

FIG. 6 illustrates a flowchart corresponding to a third step, according to various aspects of the current invention.

FIG. 7 illustrates the interface of the third step.

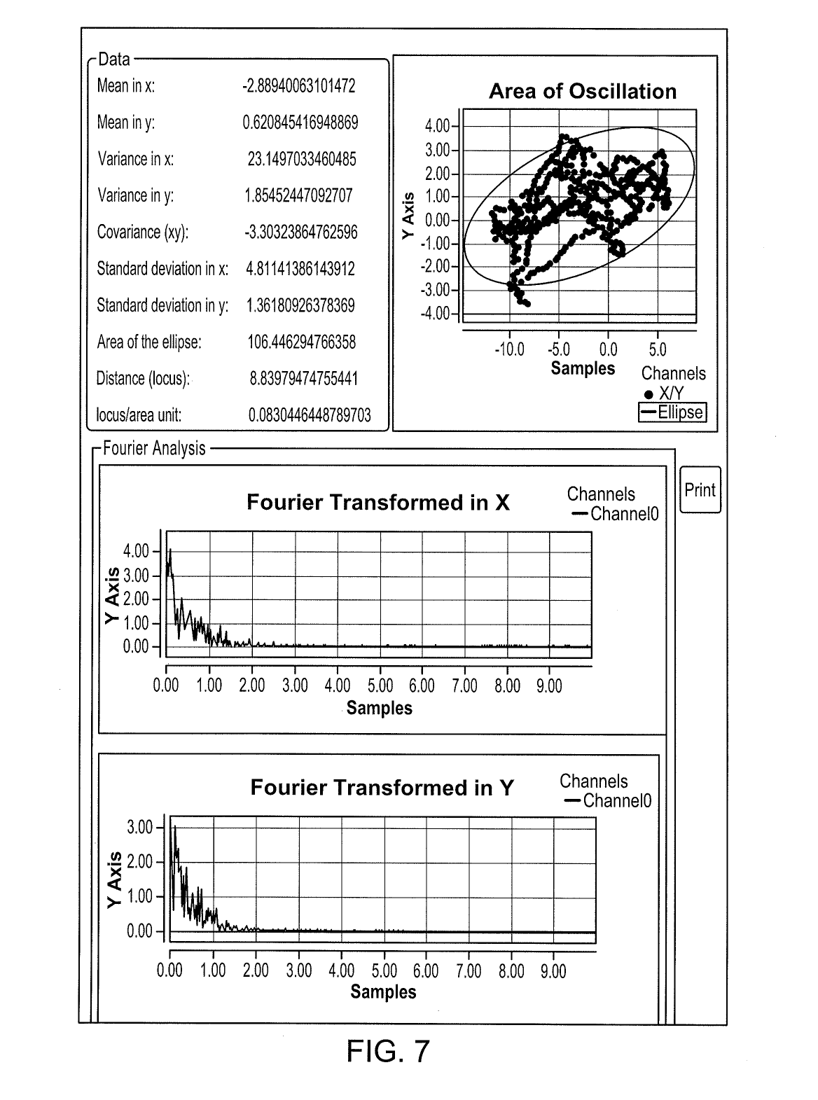

FIG. 8 illustrates a flowchart corresponding to the results step, according to various aspects of the current invention.

FIG. 9 presents an example system diagram of various hardware components and other features, for use in accordance with an aspect of the present invention.

FIG. 10 is a block diagram of various example system components, in accordance with an aspect of the present invention.

FIG. 11 is a graph illustrating the mean in the x direction and in the y direction in a first set of test.

FIG. 12 is a graph illustrating the mean in the x direction and in the y direction in a second set of test.

### DETAILED DESCRIPTION OF THE INVENTION

The aspects of the present invention are described with more detail below with reference to the attached schemes, wherein the variations and the aspects of the present invention are shown. The aspects of the present invention may, however, be carried out in many different ways and are not to be construed as limited to the variations set out herein, but the variations are provided such that this description is total and complete in the illustrative implementations, and the scope of the same is completely transmitted to those skilled in the art.

Unless otherwise defined, all the technical and scientific terms used in this document have the same meaning as commonly understood by a skilled in the art to which the aspects of the present invention belong. The systems and examples provided herein are only illustrative and are not intended to be limiting.

As long as the mathematical models are capable of reproducing figures reported in experiments, they can still be considered for modelling various natural processes.

The functioning of the Wii balance board (Wii Balance Board), comprises the following steps:

a) Basic characteristics.
b) Internal mechanism.
c) Means of communication.

a) Basic characteristics: The balance platform emerges as an entertainment system of the Wii entertainment system, created under the Japanese company Nintendo©, on which the user places the feet, and when doing this the platform emits information such as the Body Mass Index (BMI) (Nintendo 2008); and its relevant characteristics are:

Maximum supported weight 150 Kg.

4 pressure sensors.

Data transmission via Bluetooth.

b) Internal mechanism: The platform uses multiple sensors to fulfil its purpose. For example, if a person leans towards the left, exerts pressure on the left side of the platform and the sensors are responsible for detecting and sending the weight variation (Peek 2008).

c) Means of communication: The means through which the platform communicates is via Bluetooth, this is a transmission mechanism by radio frequency links, using the Bluetooth Services Discovery Protocol (SDP). In this way when the computer sends a request to the Bluetooth devices within its range, the platform sends a block of information to provide its specifications to the computer, and from there the connection is established.

d) Data Format: The Wii Balance Board reports its information as 8 bytes of data, which are read from the address 0xa40008 and is transmitted via Data Reporting Mode including extension bytes. The first 8 bytes contain the following information:

As shown in Table 1, the Wii Balance Board sends 16 bits of data for each of the four pressure sensors, along with the calibration data necessary for handling conversions to mass measurements.

TABLE 1
Data format of the sensors.

Bit

| | | | | | | | | |
|---|---|---|---|---|---|---|---|---|
| Byte | 7 | 6 | 5 | 4 | 3 | 2 | 1 | 0 |

| | |
|---|---|
| 0 | Top Right<15:8> |
| 1 | Top Right<7:0> |
| 2 | Bottom Right<15:8> |
| 3 | Bottom Right<7:0> |
| 4 | Top Left<15:8> |
| 5 | Top Left<7:0> |
| 6 | Bottom Left<15:8> |
| 7 | Bottom Left<7:0> |

The information to calibrate the sensors is sent in 24 bytes, as shown in Table 2, which contain values for the four sensors at different weights. To calculate the weight in each sensor, an interpolation among the calibration values including the reading is carried out, and the total weight in the table is the sum of these values.

TABLE 2
Data format of the sensors.

Bit

| | | | | | | | | |
|---|---|---|---|---|---|---|---|---|
| Byte | 7 | 6 | 5 | 4 | 3 | 2 | 1 | 0 |

| | |
|---|---|
| 0 | Top Right 0 kg value<15:8> |
| 1 | Top Right 0 kg value<7:0> |
| 2 | Bottom Right 0 kg value<15:8> |
| 3 | Bottom Right 0 kg value<7:0> |
| 4 | Top Left 0 kg value<15:8> |
| 5 | Top Left 0 kg value<7:0> |
| 6 | Bottom Left 0 kg value<15:8> |
| 7 | Bottom Left 0 kg value<7:0> |
| 8 | Top Right 17 kg value<15:8> |
| 9 | Top Right 17 kg value<7:0> |
| 10 | Bottom Right 17 kg value<15:8> |
| 11 | Bottom Right 17 kg value<7:0> |
| 12 | Top Left 17 kg value<15:8> |
| 13 | Top Left 17 kg value<7:0> |
| 14 | Bottom Left 17 kg value<15:8> |
| 15 | Bottom Left 17 kg value<7:0> |
| 16 | Top Right 34 kg value<15:8> |
| 17 | Top Right 34 kg value<7:0> |
| 18 | Bottom Right 34 kg value<15:8> |
| 19 | Bottom Right 34 kg value<7:0> |
| 20 | Top Left 34 kg value<15:8> |
| 21 | Top Left 34 kg value<7:0> |
| 22 | Bottom Left 34 kg value<15:8> |
| 23 | Bottom Left 34 kg value<7:0> |

Once known the operation mechanism of the platform, it was proceeded to investigate a language suitable for the needs of the project. In this process were found libraries specialized in the control of Wii components, which are developed for the Visual Basic and C# languages.

e) WiimoteLib Library: The WiimoteLib library developed by Brian Peek establishes the connection between the computer and the platform. When the Balance Board is paired, it is registered as a Human Interface Device (HID) so the Win32 Application Programming Interfaces (APIs) are used for management of HID devices (Peek 2008).

With the HID devices, the data are sent and received as reports. In other words, it is a data buffer of a predefined size with a header that determines the type of report sent. Since the data are constantly sent and received it is necessary to use asynchronous input and output operations.

The present invention comprises 3 steps or modules, which are:

1) Data capture;

### 2) Visualization; and

3) Mathematical analysis.

Wherein, the step or module 1; concerning the data capture consists of the characterization of the sensors, as well as the obtaining of the data produced by these. It also consists of a custom log file, for storage and further analysis.

The medical record is a useful tool for any diagnosis device, because it allows a simple management for the subject data. The first section of the system consists of creating this medical record with information that may be of importance not only for the physician, but also for evaluating a person.

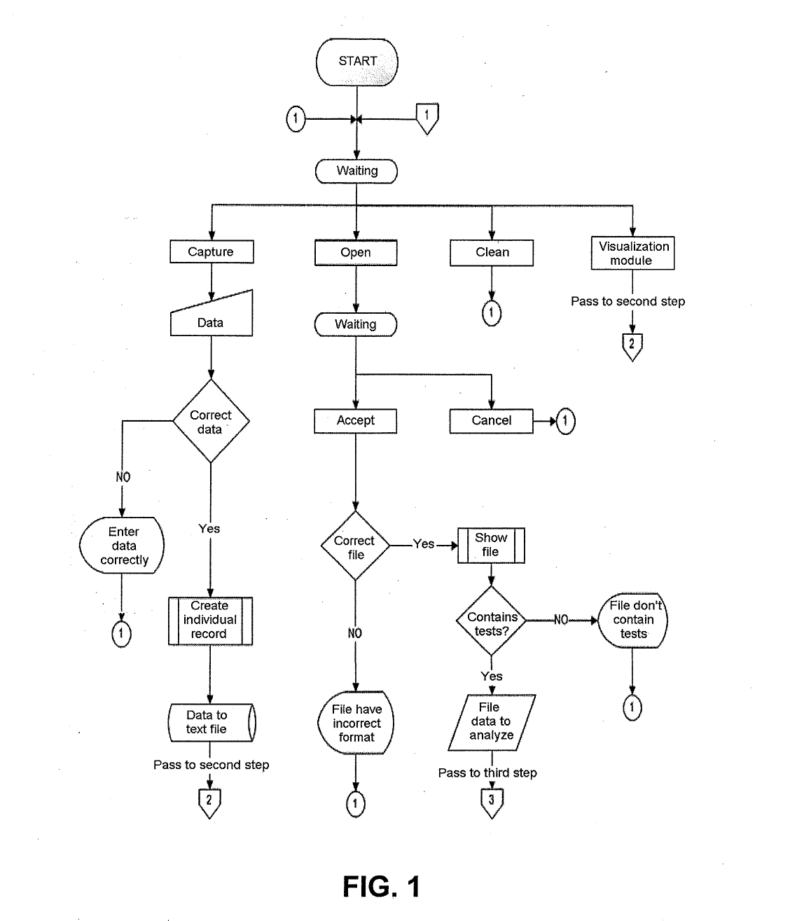

FIG. 1 shows the functioning of the step or module 1, and which has the following elements:

### Module 1.1: Subject Data

—Name: The full name of the subject. When creating the medical record, the system takes the initials of the person to create the name of the log. The examiner may not be able to create the file unless this field is full.

—Age: The age of the subject in numbers. The system checks that the entered age is a valid number, otherwise, it displays a message alerting the user of said failure. This field is of importance since it has been proven that the age is an important factor for damage related to the vestibular system (Herdman 1997).

—Height: The height in metres of the subject. In the same way as in the “Age” field, the system checks that the entered age is a valid number, otherwise it alerts the user of said failure.

—Weight: The weight in kilograms of the subject. This value is not entered by the user. The balance platform takes the information from the sensors at the time of creating the file to provide the exact weight of the person, which same data may be used during the testing of the next module.

—Sex: The gender of the subject. The examiner may select the corresponding checkbox, either (F) for female, and (M) for male. This value cannot be empty.

—Observations: Where necessary, the user of the system may be able to enter additional information about the subject before performing the test. This field is useful to describe diseases or background with medical relevance, and as well as the age field, it is important to create a more accurate diagnosis of the results obtained in the following modules.

### Module 1.2: Examiner Data

—Name: The full name of the user. This field is important because if the system is used by different doctors, the field may serve to differentiate the individual records, and group them for easy handling. Each time a file is created, the record is stored in a sub folder sorted by date within a separate folder for the examiner. In the event that no name is entered at the moment of capturing the data, the system may generate a folder called “Anonymous Examiner”.

### Module 1.3: Name of the File

This field allows to create an identifier for the records. The user has up to three letters or numbers to generate a unique prefix associated with a set of files. Once said prefix has been selected, the system may generate a count-up for each file within the set, starting with the value “0001”.

### Module 1.4: Dossier

—Name: Once the file has been generated, the name (the three characters of the prefix, the four digit count-up and the initials of the name and surname of the subject) may be displayed in this field. The name of the file cannot be modified.

—Date of issue: Day, month and year in which the record was created. This field shows the date (corresponding with the date of the computer on which the program is running) in which the file of the subject was generated. In the same way as the previous field, this information cannot be modified.

—Time of issue: Moment in which the record is created. This field uses the time that corresponds to the computer running the program, and which, in turn, is shown in the upper right corner of the system below the module “Current time”.

### Module 1.5: Samples Obtained

—Total: Number of samples captured by the system during the stability tests. This field may only take two values: 512, in the event that the time of the test has been programmed for thirty seconds, or 1024, in the event that the time of the test is of one minute.

—Frequency: Relates to the frequency of sampling of the system. By operation of the International Society of Posturography, a frequency of 20 Hz has been designated, since a human cannot oscillate to a frequency greater than 10 Hz (Kapteyn et al. 1983).

Even so, the user can choose to sample at a frequency greater than 40 Hz if desired, selecting the box of high frequency (40 Hz) within the system and before starting the test.

### Module 1.6: Other Options

—Capture: When selecting this option, the user may generate the file corresponding to the subject, the same in which the results of the test may be saved. If the fields of modules A, B and C are properly assigned, the system creates the personal medical record and displays the resulting information in module D. In case of an error, the corresponding messages are displayed so that the user can continue with the capture. While a file is opened through the “Open” option, this button may remain disabled.

—Clear: The examiner can use this option to reset the values for all the fields, facilitating the capture in the event of an error.

—Open/Close: Using this button, the user can access previous records. This option allows analyzing the results for a better feedback of the rehabilitation of the subject. The record information appears in a new window, along with the mathematical analyses applied to the said subject tests. The examiner can open any number of files, or else, start the tests with the files open to be compared. After a file is opened, the text may change to “Close”, which allows the physician to continue with the capture of new files.

### Module 1.7: Monitoring of the Sensors in the Platform

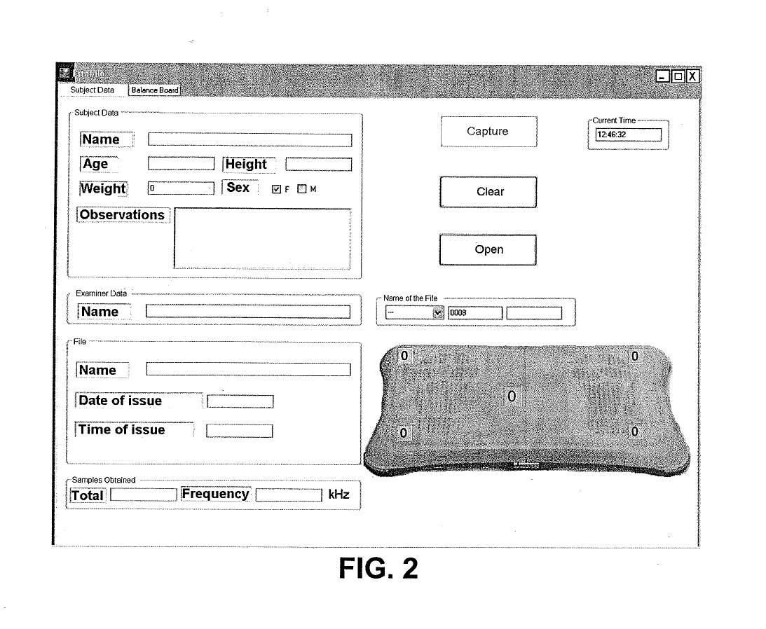

Finally, in this section there is a view of the platform and of each of the four sensors handled. The weight that is being applied to the respective sensor is shown in the external fields, while in the central field, the total weight of the person is shown. Due to the sensitivity of the platform, it is possible that the values oscillate constantly even when the subject does not have an apparent motion. FIG. 2 shows the interface of FIG. 1 and the data to be captured can be observed, as well as the various options that the Examiner has to create a new record, or to analyze a previously made test.

The movement of the body while standing in one direction, either front/back, or either sideways, can be represented as a function of time. This representation is called Stabilogram (or Sbg). Within this model, the timeline is handled horizontally, and the front and right movements of the body are handled as the positive part of the vertical axis.

Another way in which the movement of the body can be represented is as displacements of the centre of pressure of the body through the platform. This type of representation is called Statokinesigram (or Skg). In this model, the lateral movements must may be associated with the x-axis, the swings to the right being the positive side, while the front/back movements are associated to the y-axis, the front oscillation being the positive part. (Kapteyn et al. 1983).

The step or module 2; referring to the visualization, comprises the visualization of the data obtained through the stabilometric tests. The data are represented in a statistical manner, for future analysis, and graphically to facilitate the understanding of the examiner. The visualization of the data in the graphics was made to give a facilitated understanding of the movement of the individual within a time interval to the physician or examiner. This also provides additional data such as for example the mean or the average of the data that may be useful in the third step.

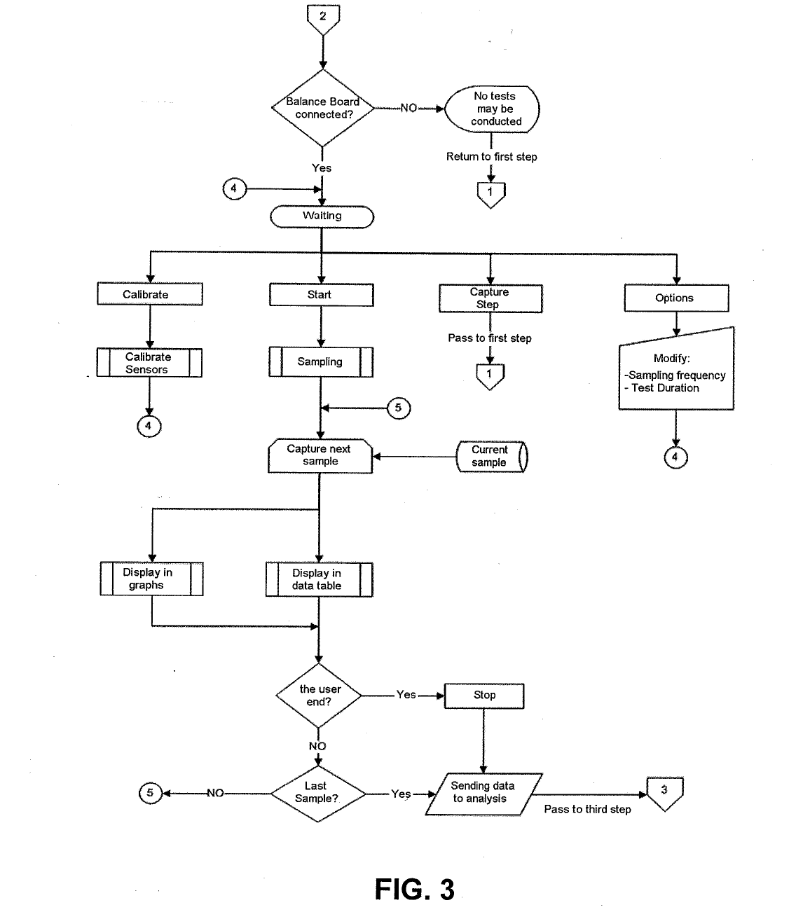

FIG. 3 shows the functioning of the step or module 2, and which has the following elements:

### Module 2.1: Monitoring of the Sensors in the Platform

This method is the easiest to visualize the movements of a person. The values are handled as integers for each of the four sensors handled by the platform. The weight that is being applied to the respective sensor is shown in the external fields, while in the central field, the total weight of the person is shown. Due to the sensitivity of the platform, it is possible that the values oscillate constantly even when the subject does not have an apparent motion.

### Module 2.2: Functions

—Calibrate: The composition of the platform causes the sensors to have different zero values even if there is not a weight located on them. The interference and the noise by the communication channel may affect the data received from the same so it is necessary a calibration prior to the analysis of a subject. This option attempts to remove the initial values and the interference (white noise) that is obtained from the board, so that more accurate data during the test may be obtained. It is important to highlight that even after the calibration process, the platform continues receiving low values in its sensors.

—Start: By selecting this option, the test may begin. Prior to this, the person may be placed on the board, following the instructions given in the annex A. The monitoring of the subject is carried out during 30 seconds or 60 seconds, depending of the selected option. Meanwhile, the examiner can follow-up through the various graphs that are presented in this module.

—Stop: The system automatically ends the test after the selected time period, but if the user so wishes, they can finish the examination by pressing this button. When doing this, the important mathematical analyses and data are shown in a new window, corresponding to the third module of the system.

### Module 2.3: Options for the Test

—Duration: 30 or 60 seconds, according to what the user wants. Since there is not a time standard for the tests, the most common options were used for this system, according to studies made (Kapteyn et al. 1983).

—Sampling frequency: 20 Hz or 40 Hz, depending on the user. The frequency of 20 Hz is the minimum required to detect oscillation frequencies approaching the 10 Hz. Even if a person does not oscillate at greater frequency, the second option (40 Hz) gives a higher resolution to the test.

### Romberg Test

The Romberg test is commonly applied during a neurological examination to assess the integrity of the dorsal columns of the spinal cord. It has evolved into a valuable clinical tool. This test provides an important key for the presence of pathologies in the proprioceptive channel and it may be carried out in a meticulous manner during the neurological assessment (Khasnis and Gokula 2003).

—Type of Test:

1. Romberg with eyes open: This test assesses the stability of the subject, while making use of their three systems (visual, proprioceptive and vestibular). The subject removes their shoes and matches the feet on the tracks marked on the platform. With the arms at the sides and with eyes looking forward on a fixed point, they try not to sway during the entire test.
2. Romberg with eyes closed: This test assesses the stability of the subject, while the visual system is disturbed. The subject removes their shoes and matches the feet on the tracks marked on the platform. With the arms at the sides, and the eyes closed, the subject tries not to sway during the entire test. In this test it is recommended to use a black mask to prevent the subject from opening the eyes, either for fear of falling, or to mislead the test (Kapteyn et al. 1983).
3. Romberg on foam with open eyes: This test assesses the stability of the subject, while the proprioceptive system is disturbed. The subject removes their shoes and is situated on a foam cushion, so that it is positioned on the platform. With the arms at the sides, and the eyes open, the subject tries not to sway during the entire test. It is recommended to locate the platform close to a wall or that an assistant stays behind the subject during the entire test, to avoid a fall.
4. Romberg on foam with eyes closed: This test assesses the stability of the subject, while the proprioceptive system and the visual system are disturbed, in such a way that they have to be based on the vestibular information to orient themselves in the space. The subject removes their shoes and is situated on a foam cushion, so that it is positioned on the platform. With the arms at the sides, and the eyes closed, the subject tries not to sway during the entire test. The same recommendations made for the previous tests should be followed.

as the analysis progresses, the examiner may be selecting the various tests that together serve to evaluate the systems that make up the posture.

### Module 2.4: Analysis Table

This data table shows the values captured by the sensors while the test is running. Every time a sample is taken, its value (expressed with precision of 6 decimal places) is added to this table. The first two columns are associated with the x (lateral movement) and y (front/back movement) axes, while the third column reflects the angle of oscillation.

We calculate the value of x by adding the values of the right sensors and subtracting from this result the values of the left sensors. For y, we add the values of the upper sensors, and subtract the sum of the values of the lower sensors.

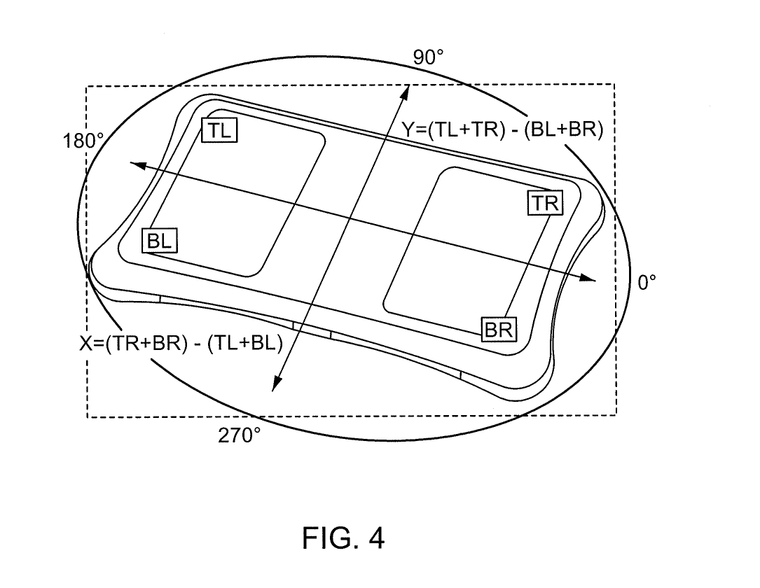

To obtain the angle of inclination of the individual being tested with respect to the plane of the Balance Board (FIG. 4) we calculate the inverse tangent of y/x, what gave us an angle in radians, and then we transformed it to degrees. In the event that the values of the upper left and upper right sensors are equal, and also the values of the lower left and right sensors are equal then it may be said that the individual is balanced in y.

If the value of x is zero (the sum of the left sensors is equal to the sum of the right sensors) then it may be balanced in x. And finally if the individual can be balanced both in x and y, the person may be completely balanced.

The equation for calculating the position of the centre of pressure as coordinates for the value of x is (Cuesta and Lema 2009):

$$X = \frac{[ ( {T}_{R} + {B}_{R} ) - ( {T}_{L} + {B}_{L} ) ] * ( \frac{Platform\_width}{2} )}{F}$$

Wherein F=T_{R}+B_{R}+T_{L}+B_{L} and Platform_Width is the size in cm of the width of the board, which in this case is 51.1 cm.

The equation for calculating the position of the centre of pressure as coordinates for the value of y is:

$$Y = \frac{[ ( {T}_{R} + {T}_{L} ) - ( {B}_{R} + {B}_{L} ) ] * ( \frac{Platform\_Length}{2} )}{F}$$

Wherein F=T_{R}+B_{R}+T_{L}+B_{L} and Platform_Length is the size in cm of the length of the board, which in this case is 31.6 cm.

These values are represented as displacements in cm, allowing important further analysis, such as the total travelled distance, or the maximum front/back and medium lateral displacement.

### Module 2.5: Stabilograms and Statokinesigram

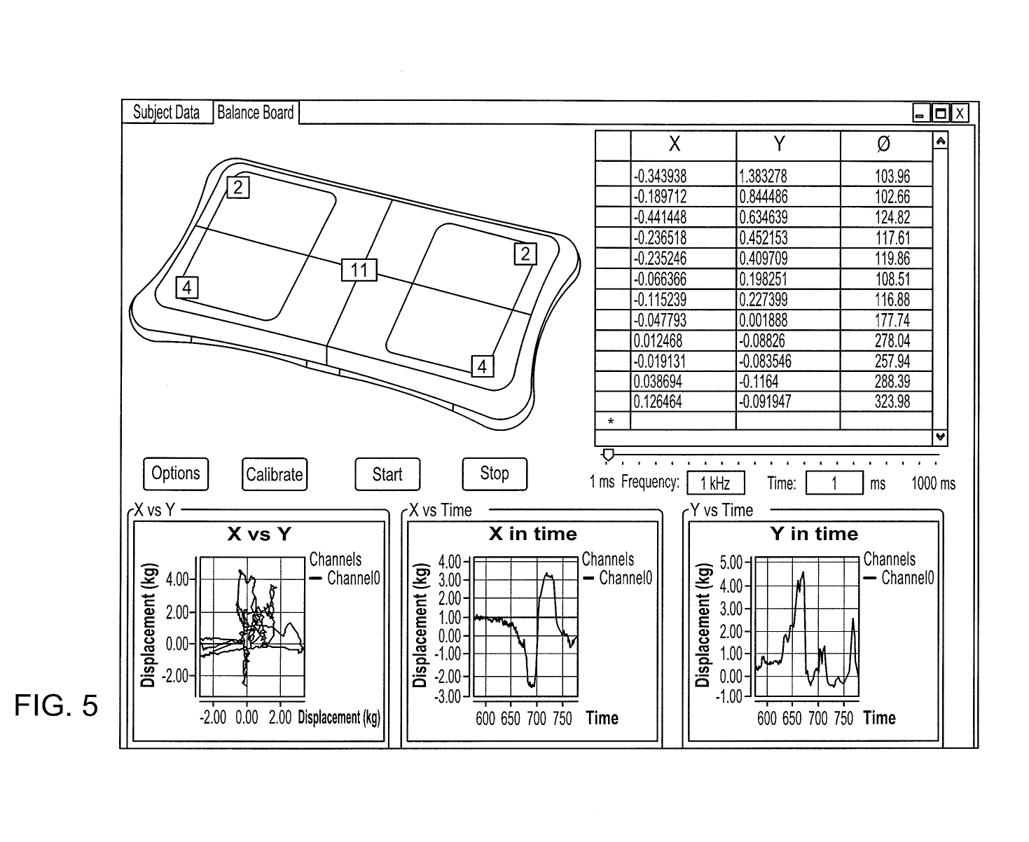

In FIG. 5. we can observe that the system has two stabilograms (one for the lateral movement and one for the front/back movement), as well as one statokinesigram. The data shown in the table are sent to these models for visualization. Each sample is checked with the corresponding time period (in the case of the stabilograms) or against its corresponding pair. The three graphics help the examiner to evaluate the position of the individual, and to detect irregularities before the mathematical analysis.

The first graph shows the displacement of the centre of pressure of the subject and its monitoring during the test. The second graph shows the stabilogram associated with the lateral movements. The graph is automatically adjusted to the weight displacement values, whereby the low oscillations are amplified such that the examiner can analyze them easily. Finally, the third graph is associated with the front and back movements, and it works in the same way as the previous stabilogram.

The step or module 3; referring to the mathematical analysis, comprises the mathematical development, wherein said analysis step is essential for the correct detection of the vestibular problems, so the understanding of the mathematical methods required for the processing of the data is emphasized. This section focuses mainly on the analysis of the fast Fourier transform to find the frequency of oscillations of each individual, and of the adjustment of an ellipse to the statokinesigram to obtain an estimate of the oscillation area of the subject being tested.

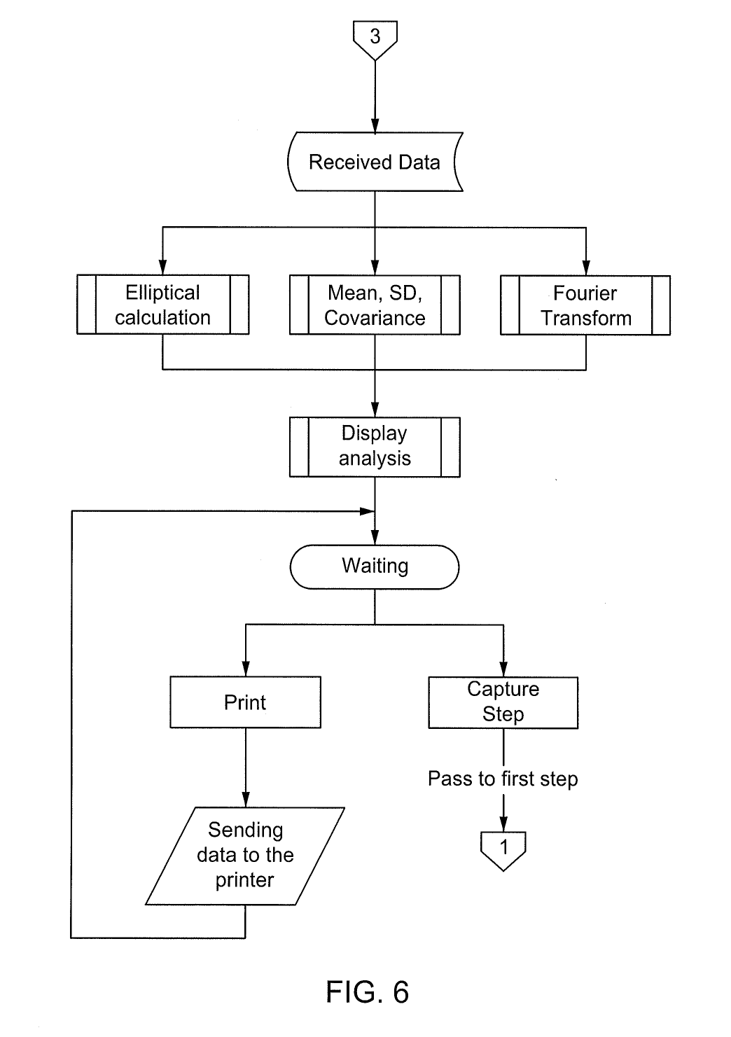

FIG. 6 shows the sequence of results of the mathematical analysis of the step or module 3, and in which are used the Fast Fourier Transform, The Discrete Fourier Series, the factorisation in sub-series, etc. As well as the following elements:

### Elliptical Adjustment

The measurement of the movement of the centre of pressure with a platform (stabilometry) is a standard procedure for the evaluation of the postural stability during rehabilitation. The subject is placed on a platform, which has pressure sensors that transmit the information through a digital analogue converter to a computer (Sevsek 2006).

From the trajectory of the centre of pressure, simple statistical parameters associated with the distance and speed are normally determined. Often, is also of interest to compare the areas inside of which the movement of the centre of pressure is confined. In this case, the analysis of the main components can be used (Oliveira et al. 1996).

In this method the eigenvalues σ₀² are calculated from the covariance matrix (σ²_{xy}):

$$( {σ}_{xy}^{2} ) = \frac{1}{N} {\sum }_{i = 1}^{N} ( x - \overline{x} ) ( y - \overline{y} ) , \qquad ( 1 )$$

wherein x̅ and y̅ are the values of the mean, while the sum is performed on the sampled points N.

Therefore, the two eigenvalues are:

σ₀²=(σₓₓ²+σ_{yy}²±\sqrt{(σₓₓ²−σ_{yy}²)²+4(σ_{xy}²)²})/2, (2)

The values of the axes of the ellipse are achieved with the square root of the eigenvalues. Since the result provides the axes of the error ellipse, it is needed to multiply by a factor to obtain the region covering 95% of the data. Therefore the value may be multiplied by 1.96 to obtain the main axes (Sevsek 2006).

The area of oscillation can then be reproduced, with an ellipse with two main axes in the θ angle (Oliveira et al. 1996):

$$tan θ = \frac{{σ}_{xy}^{2}}{{σ}_{0}^{2} - {σ}_{yy}^{2}} , \qquad ( 3 )$$

—Development of the pre diagnostic interface. Consists of the application of the methods previously studied for the development of the data. At the end of this step, it is expected to be able to successfully diagnose the problems that might exist in the examined people, or, if any medication or drug causes anomalies in the stability and posture.

### Module 3.1: Data

—Mean in x/y: The average of the data for each one of the vectors x (lateral movement) and y (front/back movement). Their values are obtained with the following equation:

$$\overline{x} = \frac{{\sum }_{i}^{N} {x}_{i}}{N} , \qquad ( 4 )$$

wherein N is the number of samples and xᵢ is the ith value in the vector.

—Standard deviation: The standard deviation of a set is the measurement of how the data are distributed. In other words, is the average distance from the mean to a point. Its equation is:

$$s = \sqrt{\frac{{\sum }_{i = 1}^{N} {( {x}_{i} - \overline{x} )}^{2}}{N}} , \qquad ( 5 )$$

wherein x̅ is the value of the mean calculated in (4).

—Variance in x/y: It is a measurement of the distribution of the data. Once again, it is calculated for the two vectors, with the following equation:

$${s}^{2} = \frac{{\sum }_{i = 1}^{N} {( {x}_{i} - \overline{x} )}^{2}}{N} \qquad ( 6 )$$

—Covariance: It is a measurement for determining how much do the vectors vary from the mean, with respect to each other. In other words, if the covariance between a vector and itself is calculated, the variance is obtained. Its value is obtained with the following equation:

$$cov ( x , y ) = \frac{{\sum }_{i = 1}^{N} ( {x}_{i} - \overline{x} ) ( {y}_{i} - \overline{y} )}{N} \qquad ( 7 )$$

—Area of the ellipse: Once the data are adjusted to the ellipse, its area is calculated, with the equation:

α=π*e₁*e₂ (8)

wherein e₁ and are the axle shafts calculated in (2).

—Line integral per second: This value is the average distance that the subject travels between two samples during the test. It is obtained by adding the distance between each sample and dividing it by the time:

$$Line / s = \frac{1}{T} {\sum }_{i = 1}^{N} \sqrt{{( {x}_{i} - {x}_{i - 1} )}^{2} + {( {y}_{i} - {y}_{i - 1} )}^{2}} \qquad ( 9 )$$

wherein T is the total time of the analysis.

Total Path=Line Integral*T (10)

—Root mean square (RMS): is the root of the quotient of the sum of the squares of the distances of the data, with respect to the mean of said data.

$$RMS = \sqrt{\frac{{\sum }_{i = 1}^{N} {( {x}_{i} - \overline{x} )}^{2} + {( {y}_{i} - \overline{y} )}^{2}}{N}} , \qquad ( 11 )$$

—Angular displacement: Due to that the displacement of the centre of pressure is influenced by the height, the angular displacement for the medial-lateral and anterior-posterior and movement is also calculated.

Knowing the maximum displacement, and the approximate height of the centre of gravity, that is obtained based on anthropometric tables, the angle of oscillation of the body is obtained.

$${θ}_{x} = \frac{180 * a tan ( \frac{d max}{h * 0 , 55} )}{π} \qquad ( 12 )$$

wherein dmax is the maximum displacement of the centre of pressure in millimetres, and h is the height of the subject (Baydal-Bertomeu et al. 2004).

—Evaluation of the proprioceptive system.—Results from the quotient of the area of the ellipse obtained during the Romberg test with eyes closed, over the area of the ellipse obtained through the Romberg test with eyes open.

$${}_{prop} S = \frac{{α}_{ECR}}{{α}_{EOR}} \qquad ( 13 )$$

The result of this equation tends to be greater than 1 if the subject uses more the information from the visual system, than the information from the proprioceptive system.

—Evaluation of the visual system.—Results from the quotient of the area of the ellipse obtained during the Romberg test on foam with eyes open, over the area of the ellipse obtained through the Romberg test with eyes open.

$${}_{vis} S = \frac{{α}_{OFR}}{{α}_{EOR}} \qquad ( 14 )$$

The result of this equation tends to be greater than 1.0 if the subject uses more the information from the proprioceptive system, than the information from the visual system.

—Evaluation of the vestibular system.—Results from the quotient of the area of the ellipse obtained during the Romberg test on foam with eyes closed, over the area of the ellipse obtained through the Romberg test with eyes open.

$${}_{vest} S = \frac{{α}_{CER}}{{α}_{EOR}} \qquad ( 15 )$$

Since the information from the visual system cannot be eliminated, the equations (13) and (14) do not give a 100% successful result, since two of the three systems responsible for the balance are being used.

### Module 3.2: Area of Oscillation

This graph is a representation of the statokinesigram, and the ellipse calculated adjusted to the data. It is an easy way to observe the calculations of the previous section, since it shows the mean for both vectors (which provides the central point of the ellipse) as well as the different elliptical areas using the values for 98.9%, 95%, 85% of the data coverage.

The “relative position” box adjusts the data with the x and y axes. Such that the values are displayed as displacement from the centre of pressure (the mean of the data is taken as the origin). The “position in relation with the board” box shows the values taking the displacement from the centre of the board towards the centre of pressure of the person.

### Module 3.3: Fast Fourier Transform

These graphs show the frequency bands associated with the oscillation of the subject. The spectrum consists of a range of 0 Hz to 10 Hz, with intervals of 0.02 Hz.

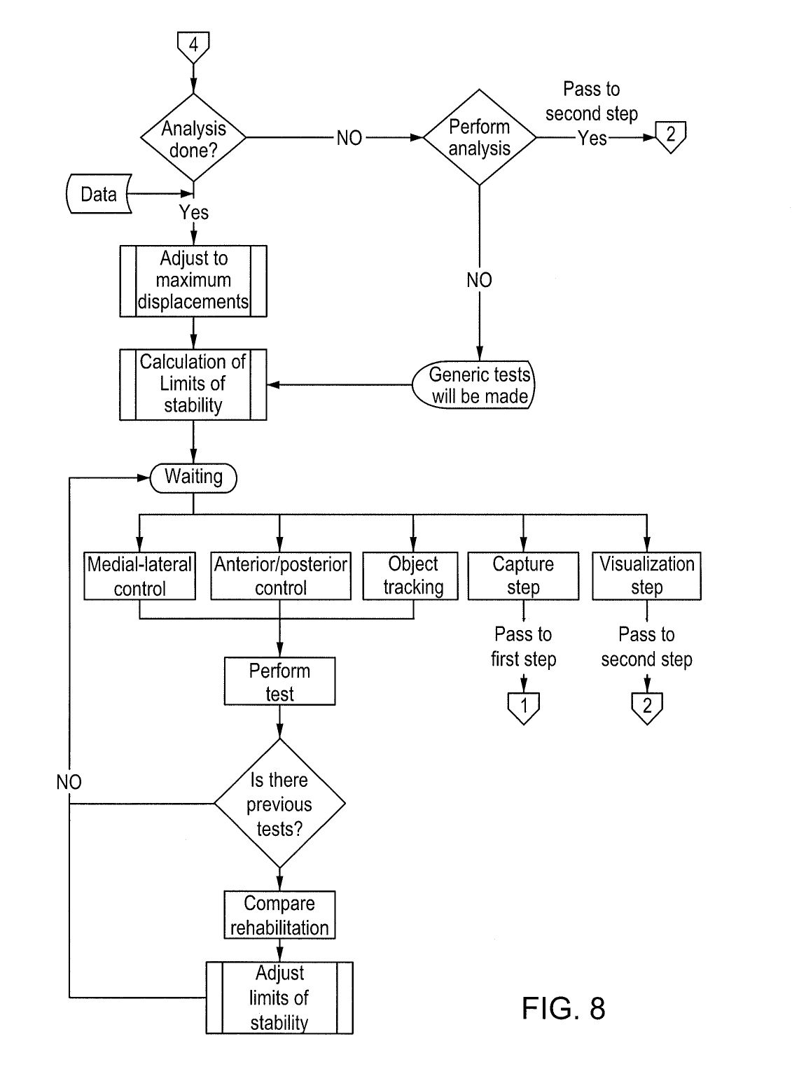

—Development of the interface of the implementation of results. In FIG. 8 we can observe that in this section the correction exercises mentioned above are implemented, such that they serve as support for the people who are suspected of having balance problems.

### Limits of Stability

The test of the analysis of the limits of stability quantifies the characteristics of the movement associated with the skill that the subject has to voluntarily change their spatial position and to maintain the stability in a new position (Baydal-Bertomeu et al. 2004).

In this test, the subject sees on a screen a cursor that represents their centre of pressure. Next, said cursor should be moved to one of the 8 targets which are placed at a distance relative to their limit of stability. (Initially, they are located outside the limit of any person, which forces the person to reach their own limits). Each target is located at 45° intervals and on each one they may stay 5 seconds.

The test evaluates the limits of stability, the reaction time of the subject for beginning their displacement, the speed of movement and the ability to control the displacement of their centre of pressure, determined by the straightness with which they move towards the targets (Garcia 2007).

### Anterior-Posterior and Medial-Lateral Control

The test of the analysis of the rhythmic and directional control is based on the follow-up of a moving target located on the screen. This test describes the characteristics of the movement associated with the skill that the subject has to change their spatial position from right to left and from front to back in a rhythmic way. The distance travelled by the subject is 60% of the maximum distance calculated in the test of the limits of stability (Baydal-Bertomeu et al. 2004).

In this exercise, the subject moves their centre of gravity, following the target, which moves at different speeds in the anterior-posterior and the medial-lateral axis. The target moves at three different speeds (increasing as time progresses) and the speed at which the person is able to move the centre of pressure, as well as the control they have to do it is evaluated (Garcia 2007).

—Testing of the application. Once completed the system, there were carried out sufficient tests to detect failures in the system, corrections and adjustments to the rules necessary for the implementation of the system in any medical institution. With the purpose of creating a control group, the stability of various subjects was analyzed and in this way the system was calibrated, and thus a control pattern for the population of the studied age was generated.

### Example 1

### Study of the Normal Balance in the Young Healthy Population (Test 1)

The first step performing the battery of Romberg tests on a number of people (Table 3) within the 20 to 30 age group; 12 men and 5 women were evaluated, following various specifications.

1.—The subjects removed their shoes before starting the analysis, and placed their feet on the footprints marked on the platform. The separation of the heels was approx. 2 cm. The angle of inclination of the feet was 30°.

2.—Each one is instructed to see forward, with the arms at the sides, focusing their gaze at a fixed point at an approximated distance of one metre. They were also told to try to swing as little as possible.

3.—The tests were carried out in a closed room, with low noise, with the platform located at a 1 meter distance from the wall.

4.—The duration of each test was 30 seconds, with a sampling frequency of 40 Hz.

5.—During the Romberg tests with eyes closed, the subjects were instructed to not open the eyes until they were told otherwise, due to the lack of a mask.

6.—During the Romberg tests on foam, an examiner stood near the subjects, to prevent falls or to hold them in the case of help.

7.—The wait time between each test was 10 seconds, this is to avoid that the subjects became accustomed to the exercises.

TABLE 3
Data of the subjects for the control group 1

| Subject | Age | Height | Weight | Sex |
|---|---|---|---|---|
| 1 | 23 | 1.83 | 97 | M |
| 2 | 23 | 1.67 | 89 | M |
| 3 | 28 | 1.66 | 61 | M |
| 4 | 22 | 1.80 | 72 | M |
| 5 | 23 | 1.85 | 77 | M |
| 6 | 20 | 1.72 | 79 | M |
| 7 | 32 | 1.52 | 65 | F |
| 8 | 23 | 1.72 | 51 | M |
| 9 | 22 | 1.73 | 82 | M |
| 10 | 21 | 1.63 | 71 | F |
| 11 | 22 | 1.67 | 77 | F |
| 12 | 23 | 1.65 | 63 | M |
| 13 | 22 | 1.52 | 45 | F |
| 14 | 23 | 1.84 | 79 | M |
| 15 | 22 | 1.60 | 75 | F |
| 16 | 23 | 1.68 | 62 | M |
| 17 | 23 | 1.93 | 62 | M |

The variables that were highlighted for creating the control group were: the mean in x (for the four tests), the mean in y, the area of the ellipse (once more for the four tests), the anterior-posterior and medial-lateral maximum displacement, the angular displacement and the most significant frequency bands according to Fourier analysis.

### Evaluation of the Mean in x and in y

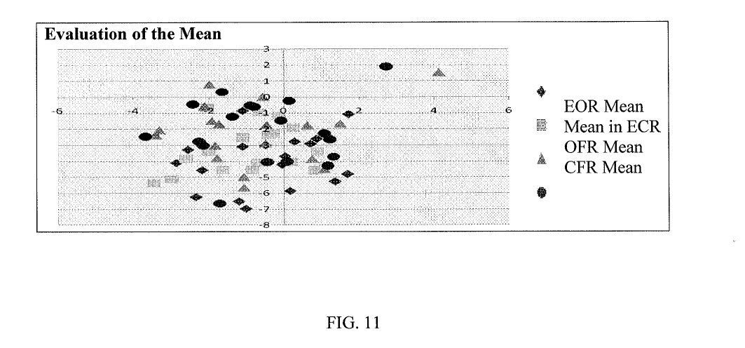

The results of the four tests are shown in FIG. 11.

The graph illustrated in FIG. 11 shows that in the comparison of the four tests the value of the OFR and CFR tests move away from the centre of the graph.

### General Evaluation

According to the results obtained, it can be seen that the means in x and y vary greatly, both among the subjects and between the tests. The mean in y for each subject is usually negative, which is an indication that the persons exert more pressure on the heels than on the tips of the feet. This is explained by the very shape of the feet. The maximum displacements in x are smaller compared to the maximum displacements in y, confirming the data mentioned before. This also indicates that the persons oscillate more in an anterior-posterior manner, than in a medial-lateral manner.

As the test increase in difficulty, the displacements increase, and it can be seen that the mean in y is closer to 0 cm, which means that in order to try to compensate for the lack of balance, the people move their weight forward.

These tests confirm that the balance of a people is better when they use the three systems (visual, proprioceptive and vestibular) than when they use only two. While maintaining the balance, it may not be compensated or kept in the same way than when it is in full use of the information related with these systems.

The line integral per second indicates the rate of change between distance and time, and it is an indication of the transitions that each subject carried out during the tests. Even if the area of oscillation is small, the integral line per second can detect oscillations, or else, the average transitions that were carried out during each test. Finally, the distance travelled, shows the full path that the centre of pressure of each individual followed during the test.

TABLE 4
Results of the EOR test.

| | Romberg tests with eyes open | | | | | |
|---|---|---|---|---|---|---|
| | | | | | (cm/s) | |
| | (cm) | (cm) | (cm) | (cm) | line | (cm) |
| | Mean | Mean | Max. | Max. | integral | Distance |
| Subject | in x | in y | Displ. in x | Displ. in y | per s. | Travelled |
| 1 | 1.707 | 1.064 | 3.137 | 4.711 | 2.6553 | 67.7229 |
| 2 | −2.188 | −4.539 | 0.642 | 1.059 | 1.748 | 44.574 |
| 3 | −1.104 | −0.85 | 1.949 | 1.837 | 2.36 | 60.18 |
| 4 | 0.288 | −2.764 | 1.956 | 2.487 | 2.14 | 54.57 |
| 5 | 0.865 | −2.597 | 2.042 | 4.453 | 2.703 | 68.9265 |
| 6 | −2.867 | −4.084 | 1.23 | 1.752 | 2.105 | 53.6775 |
| 7 | −1.199 | −6.495 | 1.368 | 1.956 | 2.245 | 57.2475 |
| 8 | 1.676 | −4.787 | 1.335 | 1.336 | 2.86 | 72.93 |
| 9 | 0.701 | −2.905 | 1.509 | 2.322 | 1.939 | 50.7195 |
| 10 | −1.018 | −6.959 | 1.438 | 2.372 | 2.492 | 63.546 |
| 11 | −2.346 | −6.224 | 2.769 | 1.SC5 | 2.126 | 54.213 |
| 12 | −1.107 | −3.072 | 1.468 | 1.649 | 2.303 | 58.7265 |
| 13 | 0.031 | −3.681 | 1.953 | 2.51 | 3.323 | 84.7365 |
| 14 | −0.49 | −2.934 | 1.178 | 2.014 | 2.132 | 54.366 |
| 15 | 0.169 | −5.851 | 1.547 | 1.875 | 2.443 | 62.2965 |
| 16 | −0.049 | −4.191 | 1.364 | 2.359 | 2.574 | 65.637 |
| 17 | −1.961 | −2.633 | 1.357 | 1.738 | 2.809 | 71.6295 |

TABLE 5
Results of the ECR test.

| | Romberg tests with eyes closed | | | | | |
|---|---|---|---|---|---|---|
| | | | (cm) | | (cm/s) | |
| | (cm) | (cm) | Max. | (cm) | line | (cm) |
| | Mean | Mean | Displ. | Max. | integral | Distance |
| Subject | in x | in y | in x | Displ. in y | per s. | Travelled |
| 1 | −0.138 | −1.178 | 2.585 | 4.367 | 3.183 | 81.1665 |
| 2 | −2.001 | −3.4 | 1.418 | 2.87 | 2.692 | 68.646 |
| 3 | −0.662 | −0.978 | 2.282 | 2.701 | 3.174 | 80.937 |
| 4 | −0.297 | −2.266 | 1.3 | 2.071 | 2.532 | 64.566 |
| 5 | 0.881 | −3.363 | 3.642 | 4.004 | 3.685 | 93.9675 |
| 6 | −2.613 | −3.815 | 1.938 | 2.323 | 2.062 | 52.581 |
| 7 | −2.99 | −5.088 | 0.797 | 2.999 | 2.574 | 65.637 |
| 8 | 1.114 | −4.244 | 1.024 | 1.209 | 2.758 | 70.329 |
| 9 | 0.25 | −1.921 | 1.127 | 2.267 | 2.19 | 55.845 |
| 10 | −0.834- | −4.531 | 1.285 | 4.205 | 3.732 | 95.166 |
| 11 | −3.454 | −5.36 | 2.866 | 3.835 | 2.802 | 71.451 |
| 12 | −404 | −2.371 | 1.191 | 1.35 | 2.408 | 61.404 |
| 13 | 0.22 | −4.031 | 2.148 | 2.363 | 3.476 | 88.638 |
| 14 | −1.088 | −2.519 | 1.881 | 3.233 | 2.592 | 66.096 |
| 15 | −0.7 | −4.049 | 3.319 | 2.572 | 2.857 | 72.8535 |
| 16 | −1.635 | −4.54 | 2.107 | 3.335 | 2.854 | 72.777 |
| 17 | −1.915 | −1.482 | 1.504 | 2.265 | 2.717 | 69.2835 |

TABLE 6
Results of the OFR test.

| | Romberg tests with eyes open. using foam | | | | | |
|---|---|---|---|---|---|---|
| | | | | | (cm/s) | |
| | (cm) | (cm) | (cm) | (cm) | line | (cm) |
| | Mean | Mean | Max. | Max. | integral | Distance |
| Subject | in x | in y | Displ. in x | Displ. in y | per s. | Travelled |
| 1 | −0.453 | −1.74 | 5.838 | 7.383 | 3.802 | 96.951 |
| 2 | −1.721 | −1.69 | 1.313 | 3.418 | 2.202 | 56.151 |
| 3 | −1.979 | 0.798 | 2.116 | 2.853 | 2.567 | 65.4585 |
| 4 | −2.11 | −0.567 | 1.358 | 1.946 | 1.949 | 49.6995 |
| 5 | 1.49 | −1.654 | 2.015 | 3.083 | 2.249 | 57.3495 |
| 6 | −3.406 | −2.351 | 1.645 | 1.728 | 2.05 | 52.275 |
| 7 | −3.309 | −2.047 | 1.197 | 2.826 | 2.113 | 53.8815 |
| 8 | 0.629 | −1.761 | 1.123 | 2.053 | 2.702 | 68.901 |
| 9 | −0.486 | −2.865 | 0.86 | 1.962 | 1.829 | 46.6395 |
| 10 | −1.776 | −3.783 | 1.497 | 2.008 | 2.198 | 56.049 |
| 11 | −1.06 | −5.614 | 1.771 | 2.051 | 2.152 | 54.876 |
| 12 | −0.56 | 0.01 | 1.28 | 1.896 | 2.25 | 57.375 |
| 13 | 0.754 | −3.825 | 2.28 | 2.557 | 3.272 | 83.436 |
| 14 | 4.117 | 1.541 | 2.046 | 2.281 | 2.414 | 61.557 |
| 15 | −1.831 | −3.014 | 1.308 | 1.788 | 2.105 | 53.6775 |
| 16 | −1.607 | −4.955 | 2.218 | 3.316 | 2.826 | 72.053 |
| 17 | −0.891 | −0.518 | 1.942 | 1.942 | 2.319 | 2.837 |

TABLE 7
Results of the CFR test.

| | Romberg tests with eyes closed. using foam | | | | | |
|---|---|---|---|---|---|---|
| | | | | | (cm/s) | |
| | (cm) | (cm) | (cm) | (cm) | line | (cm) |
| | Mean | Mean | Max. | Max. | integral | Distance |
| Subject | in x | in y | Displ. in x | Displ. in y | per s. | Travelled |
| 1 | 0.137 | −0.227 | 3.623 | 6.295 | 3.881 | 93.9655 |
| 2 | −0.784 | −0.601 | 2.38 | 4.103 | 3.401 | 86.7255 |
| 3 | −1.639 | 0.326 | 2.199 | 3.114 | 3.022 | 77.061 |
| 4 | −2.431 | −0.442 | 1.677 | 3.571 | 2.499 | 63.7245 |
| 5 | 1.706 | −2.267 | 4.19 | 5.125 | 3.95 | 100.725 |
| 6 | −3.683 | −2.456 | 1.953 | 2.577 | 2.618 | 66.759 |
| 7 | −2.258 | −2.758 | 1.716 | 3.255 | 2.904 | 74.052 |
| 8 | 1.224 | −2.629 | 1.833 | 3.127 | 2.949 | 75.1995 |
| 9 | 0.085 | −4.001 | 1.576 | 2.679 | 2.225 | 56.7375 |
| 10 | −2.156 | −3.004 | 1.763 | 3.811 | 3.101 | 79.0755 |
| 11 | −1.721 | −6.621 | 1.662 | 3.76 | 2.543 | 64.8465 |
| 12 | −0.081 | −1.467 | 0.844 | 1.799 | 2.497 | 63.6735 |
| 13 | 1.172 | −4.275 | 2.01 | 2.414 | 3.581 | 91.3155 |
| 14 | 2.704 | 1.903 | 3.05 | 4.32 | 3.09 | 78.795 |
| 15 | −1.371 | −1.212 | 2.135 | 3.129 | 2.494 | 63.597 |
| 16 | −0.443 | −4.034 | 2.599 | 5.146 | 3.843 | 97.9965 |
| 17 | −2.04 | −0.673 | 1.892 | 2.046 | 3.002 | 76.551 |

### Evaluation of the Areas of Oscillation

In accordance with the analyses carried out, several subjects obtained a smaller area of oscillation in the Romberg test with eyes closed, in comparison with the Romberg test with eyes open (Table 8). This may be due to two things: since the tests were performed in order of difficulty, the first being the EOR test, it is possible that the subjects felt nervous or altered by the analysis. Secondly, the subjects could have been tired of staring at the selected point in the EOR test, so it is possible to they have looked elsewhere, causing a loss of concentration and balance. However, when calculating the average of the areas of oscillation it was shown that the balance is better when using all the systems related with the balance, showing that the platform and the project are able to determine changes in posture. Cuesta and Lema reported similar results (Cuesta and Lema 2009).

TABLE 8
Areas of oscillation for each of the tests.

| | Area (cm²) | | | |
|---|---|---|---|---|
| Subject | EOR Elipse | ECR Elipse | OFR Elipse | CFR Elipse |
| 1 | 5.03537184 | 5.26524917 | 8.88437257 | 20.4191662 |
| 2 | 6.28736007 | 6.00615817 | 15.76768 | 12.176234 |
| 3 | 0.44881755 | 3.30251629 | 2.87004652 | 7.42324305 |
| 4 | 1.7359871 | 3.77779686 | 2.95065403 | 4.4261495 |
| 5 | 2.95646798 | 1.37019518 | 1.73162835 | 3.74538826 |
| 6 | 4.78257475 | 8.340898 | 6.15088654 | 13.3828214 |
| 7 | 1.73681432 | 2.72271867 | 1.85068409 | 4.21274802 |
| 3 | 1.49809168 | 1.6693281 | 2.93362076 | 3.85818829 |
| 9 | 1.28072738 | 1.16226415 | 1.90361643 | 3.22514461 |
| 10 | 1.56684803 | 2.05931085 | 1.08021856 | 3.41969391 |
| 11 | 1.59360132 | 4.00871877 | 2.1084094 | 4.249809 |
| 12 | 3.98353418 | 8.00234265 | 2.59138338 | 5.34552785 |
| 13 | 1.45464683 | 1.08594431 | 1.99071012 | 1.06482539 |
| 14 | 2.0865675 | 2.65833037 | 2.98078001 | 3.15316374 |
| 15 | 1.53519433 | 4.1111105 | 3.68012374 | 9.7108911 |
| 16 | 2.1499647 | 7.73595395 | 1.37482756 | 4.70995742 |
| 17 | 1.35363947 | 5.77712371 | 5.60013032 | 7.83775802 |
| 13 | 2.76629257 | 1.99717868 | 1.90264527 | 3.6625019 |

The mean for each of the elliptical areas were the following:

Area (cm²)

| EOR Elipse | ECR Elipse | OFR Elipse | CFR Elipse |
|---|---|---|---|
| 2.45847231 | 3.94739658 | 3.79735653 | 6.44573398 |

According to these results, it can be seen that the subjects used more the visual information, than the proprioceptive information. Finally, when suppressing two of the three systems, the body cannot maintain its balance properly, which accounts for the result of the fourth test.

### Example 2

### Study of the Normal Balance in the Young Healthy Population (Test 2)

A second test was carried out, to see if the instruction given at the time of the start of the test, would influence the result. On this occasion, the battery of Romberg tests was carried out on a total of 14 people: 7 men and 7 women, within the same 20 to 30 age group (Table 9). It was evaluated using the following set of specifications:

1.—The subjects removed their shoes before starting the analysis, and placed their feet on the footprints marked on the platform. The separation of the heels was approx. 2 cm. The angle of inclination of the feet was 30°.

2.—Each one was instructed to see forward, with the arms at the sides, focusing their gaze at a fixed point at an approximated distance of one metre. They were told to relax since it is natural that there is a certain oscillation while standing.

3.—The tests were carried out in a closed room, with low noise, with the platform located at a 1 meter distance from the wall.

4.—The duration of each test was 30 seconds, with a sampling frequency of 40 Hz.

5.—During the Romberg tests with eyes closed, a mask was placed on each subject, so that they could not make use of their visual system, preventing so that they “fooled” the system.

6.—During the Romberg tests on foam, an examiner stood near the subject, to prevent falls or to hold them in the case of help. The used foam cushion was of 35×35×10 cm.

7.—The wait time between each test was 10 seconds, this is to avoid that the subjects became accustomed to the exercises.

TABLE 9
Data of the subjects for the control group 2.

| Subject | Age | Height | Weight | Sex |
|---|---|---|---|---|
| 1 | 23 | 1.67 | 75 | F |
| 2 | 24 | 1.52 | 51 | F |
| 3 | 24 | 1.78 | 59 | M |
| 4 | 22 | 1.62 | 49 | M |
| 5 | 25 | 1.55 | 73 | F |
| S | 21 | 1.53 | 64 | F |
| 7 | 23 | 1.52 | 56 | F |
| 3 | 22 | 1.53 | 69 | M |
| 9 | 24 | 1.50 | 70 | F |
| 10 | 22 | 1.60 | 67 | F |
| 11 | 23 | 1.8 | 69 | M |
| 12 | 23 | 1.58 | 61 | M |
| 13 | 23 | 1.53 | 62 | M |
| 14 | 23 | 1.66 | 63 | M |

The same variables as those used for the control group 1 were analyzed.

### Evaluation of the Mean in x and in y

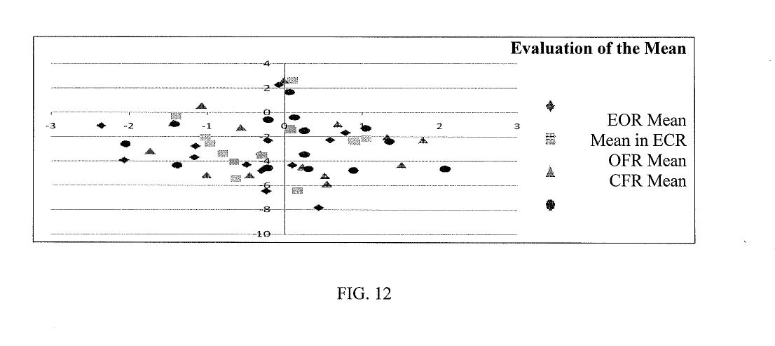

The results of the four tests are shown in FIG. 12:

It can be seen that in this occasion the data are much more distributed, but it is observed that in the ends of the graph illustrated in FIG. 12, the points are still corresponding to the high difficulty tests (OFR and CFR).

### General Evaluation

In this series of tests it was shown once again that the mean in y is usually negative. This corroborates that the system can detect the variations in weight in an appropriate manner. The maximum displacements in x were smaller than the displacements in y.

As the test increase in difficulty, the displacements increase, and it can be seen that the mean in y is closer to 0 cm, which means that in order to try to compensate for the lack of balance, the people move their weight forward.

The total travelled distance was greater when the Romberg test with eyes closed, on foam (RGO) was carried out, followed by the Romberg test with eyes closed. In this group, the results confirm that the individuals greatly depend on the visual information to maintain their balance.

Comparing with the test performed in the first control group, it is observed that the values of displacements, line integral, and travelled path are higher when using the mask. This indicates that the mask prevents the “involuntary” use of the visual system, which is why the test is more appropriate in this way. Another indication might be that the individuals are nervous when they know that they cannot open their eyes even if they feel they want to, losing concentration and oscillating more.

These tests confirm that the balance of the persons is better when they use the three systems (visual, proprioceptive and vestibular) than when they use only two. While maintaining the balance, it may not be compensated or kept in the same way than when it is in full use of the information related with these systems.

TABLE 10
Results for the EOR test.

| | Romberg tests with eyes open | | | | | |
|---|---|---|---|---|---|---|
| | | | | | (cm/s) | |
| | (cm) | (cm) | (cm) | (cm) | line | (cm) |
| | Mean | Mean | Max. | Max. | integral | Distance |
| Subject | in x | in y | Displ. in x | Displ. in y | per s. | Travelled |
| 1 | −2.06 | −3.922 | 2 | 4.221 | 3.246 | 82.773 |
| 2 | 0.787 | −1.659 | 0.838 | 2.612 | 2.602 | 66.351 |
| 3 | −0.486 | −4.288 | 1.268 | 1.97 | 2.353 | 60.0015 |
| 4 | −1.161 | −3.681 | 1.637 | 1.996 | 2.902 | 74.001 |
| 5 | −1.148 | −2.759 | 2.93 | 4.024 | 3.186 | 81.243 |
| 6 | −0.307 | −3.429 | 1.69 | 2.056 | 2.191 | 55.8705 |
| 7 | −0.23 | −6.459 | 1.901 | 2.634 | 2.844 | 72.522 |
| 8 | −0.207 | −2.308 | 2.285 | 3.414 | 2.716 | 69.258 |
| 9 | 0.105 | −4.331 | 2.26 | 4.055 | 2.545 | 64.8975 |
| 10 | 0.442 | −7.827 | 3.064 | 3.191 | 3.189 | 81.3195 |
| 11 | 0.586 | −2.255 | 1.112 | 2.013 | 2.251 | 57.4005 |
| 12 | −0.292 | −4.789 | 1.515 | 2.074 | 2.626 | 66.963 |
| 13 | −2.349 | −1.088 | 1.98 | 2.72 | 2.694 | 68.697 |
| 14 | 0.071 | 2.276 | 1.368 | 1.847 | 2.436 | 62.118 |

TABLE 11
Results for the ECR test.

| | Romberg tests with eyes open | | | | | |
|---|---|---|---|---|---|---|
| | | | | | (cm/s) | |
| | (cm) | (cm) | (cm) | (cm) | line | (cm) |
| | Mean | Mean | Max. | Max. | integral | Distance |
| Subject | in x | in y | Displ. in x | Displ. in y | per s. | Travelled |
| 1 | −0.641 | −4.076 | 1.402 | 2.665 | 2.702 | 68.901 |
| 2 | 1.052 | −2.159 | 2.273 | 1.688 | 2.862 | 72.981 |
| 3 | −0.286 | −3.527 | 1.676 | 3.123 | 2.933 | 74.915 |
| 4 | −1.019 | −2.027 | 2.426 | 3.602 | 3.184 | 81.192 |
| 5 | −0.96 | −2.573 | 1.754 | 5.149 | 3.482 | 88.791 |
| 6 | 0.888 | −2.353 | 2.656 | 2.821 | 2.347 | 59.8485 |
| 7 | −0.621 | −5.451 | 2.131 | 3.504 | 2.88 | 73.44 |
| 8 | 0.07 | −1.327 | 3.32 | 3.419 | 3.563 | 90.8565 |
| 9 | −0.796 | −3.397 | 2.093 | 5.325 | 2.65 | 67.575 |
| 10 | 0.168 | −6.402 | 4.44 | 5.456 | 3.681 | 93.8655 |
| 11 | 0.912 | −2.447 | 1.349 | 3.333 | 2.623 | 66.8865 |
| 12 | 0.084 | −1.46 | 1.459 | 3.146 | 3.055 | 77.9025 |
| 13 | −1.394 | −0.32 | 2.309 | 2.377 | 3.107 | 79.2285 |
| 14 | 0.105 | 2.617 | 1.591 | 2.356 | 2.788 | 71.094 |

TABLE 12
Results for the OFR test.

| | Romberg tests with eyes open using foam | | | | | |
|---|---|---|---|---|---|---|
| | | | | | (cm/s) | |
| | (cm) | (cm) | (cm) | (cm) | line | (cm) |
| | Mean | Mean | Max. | Max. | integral | Distance |
| Subject | in x | in y | Displ. in x | Displ. in y | per s. | Travelled |
| 1 | −0.448 | −5.163 | 1.191 | 3.464 | 2.636 | 67.218 |
| 2 | −1.433 | −0.872 | 1.296 | 2.148 | 2.626 | 66.963 |
| 3 | 0.547 | −5.886 | 2.335 | 2 | 2.79 | 71.145 |
| 4 | 0.519 | −5.217 | 1.804 | 2.412 | 2.939 | 74.9445 |
| 5 | 1.784 | −2.258 | 1.518 | 2.383 | 2.37 | 60.435 |
| 6 | 1.317 | −2.012 | 1.497 | 2.338 | 2.248 | 57.324 |
| 7 | −1.004 | −5.138 | 2.603 | 2.762 | 2.895 | 73.8225 |
| 8 | −1.731 | −3.181 | 2.316 | 2.633 | 2.638 | 67.269 |
| 9 | 0.685 | −0.953 | 1.533 | 1.779 | 2.094 | 53.397 |
| 10 | 0.228 | −4.477 | 2.573 | 3.872 | 3.177 | 81.0135 |
| 11 | −0.562 | −1.244 | 1.847 | 1.982 | 2.294 | 58.497 |
| 12 | 1.503 | −4.297 | 1.921 | 1.472 | 2.625 | 66.9375 |
| 13 | −1.07 | 0.558 | 1.92 | 2.047 | 2.819 | 71.8845 |
| 14 | −0.006 | 2.643 | 1.679 | 1.253 | 2.379 | 60.6645 |

TABLE 13
Results for the CFR test.

| | Romberg tests with eyes closed. using foam | | | | | |
|---|---|---|---|---|---|---|
| | | | | | (cm/s) | |
| | (cm) | (cm) | (cm) | (cm) | line | (cm) |
| | Mean | Mean | Max. | Max. | integral | Distance |
| Subject | in x | in y | Displ. in x | Displ. in y | per s. | Travelled |
| 1 | −0.214 | −4.541 | 2.178 | 3.951 | 3.469 | 88.4595 |
| 2 | 0.132 | −0.402 | 1.326 | 1.361 | 2.564 | 65.382 |
| 3 | 0.307 | −4.633 | 2.152 | 3.155 | 2.969 | 75.7095 |
| 4 | 0.884 | −4.75 | 1.965 | 3.049 | 3.294 | 83.997 |
| 5 | 1.349 | −2.388 | 1.37 | 3.785 | 2.866 | 73.083 |
| 6 | 1.046 | −1.306 | 1.608 | 2.208 | 2.552 | 65.076 |
| 7 | −1.381 | −4.325 | 2.268 | 3.194 | 2.988 | 76.194 |
| S | −1.414 | −0.944 | 3.564 | 4.171 | 3.571 | 91.0605 |
| 9 | −0.209 | −0.599 | 3.351 | 5.494 | 2.924 | 74.562 |
| 10 | 0.25 | −3.457 | 3.163 | 3.545 | 3.197 | 81.5235 |
| 11 | 0.255 | −1.498 | 2.659 | 2.995 | 3.252 | 82.926 |
| 12 | 2.063 | −4.622 | 4.126 | 5.094 | 3.251 | 82.9005 |
| 13 | −2.039 | −2.601 | 5.466 | 6.054 | 3.469 | 88.4595 |
| 14 | 0.066 | 1.653 | 1.59 | 3.559 | 3.012 | 76.806 |

### Evaluation of the Areas of Oscillation

Once again, several subjects obtained a smaller area of oscillation in the Romberg test with eyes closed, in comparison with the Romberg test with eyes open (Table 6), when calculating the average of the areas of oscillation it was shown that the balance is better when using all the systems related with posture. On this occasion, the test that had a smaller area of oscillation was the Romberg test with eyes open, using foam. This may be due to the constant use of the cushion through the exercises, making it lose part of its padding, and deforming until presenting little disturbance to the proprioceptive system. If we add to this the factor of habituation to the platform, then this can account for the results obtained. There may be a larger period between each test (more than 10 seconds) so the subjects do not get accustomed.

TABLE 6
Areas of oscillation for each of the tests.

| | Area (cm²) | | | |
|---|---|---|---|---|
| Subject | EOR Ellipse | ECR Ellipse | OFR Ellipse | CFR Ellipse |
| 1 | 4.71096576 | 2.63363691 | 2.36382843 | 4.34427949 |
| 2 | 1.39242575 | 0.7122946 | 1.6722333 | 1.22038908 |
| 3 | 1.59229487 | 3.22938423 | 2.7299511 | 4.38479916 |
| 4 | 2.42902008 | 4.5320527 | 2.85264674 | 3.45991665 |
| 5 | 7.95354908 | 4.2934617 | 2.05346345 | 2.79987523 |
| 6 | 2.79518759 | 5.61494168 | 2.14147974 | 2.98255357 |
| 7 | 3.61341601 | 5.01331006 | 5.10413559 | 4.99226696 |
| 3 | 5.55200609 | 8.39622779 | 5.24723064 | 11.2770911 |
| 9 | 5.01340516 | 6.78792266 | 2.22445981 | 9.2772908 |
| 10 | 5.44435074 | 12.0016804 | 5.85762677 | 5.48554324 |
| 11 | 1.58927581 | 3.92884453 | 2.16192404 | 5.87020408 |
| 12 | 1.91439431 | 2.84127141 | 2.01041006 | 6.82360646 |
| 13 | 3.81701906 | 3.59806514 | 2.86461361 | 14.5732139 |
| 14 | 1.82491556 | 1.85775224 | 1.29764984 | 2.69511269 |

The mean for each of the elliptical areas were the following:

Area (cm²)

| EOR Ellipse | ECR Ellipse | OFR Ellipse | CFR Ellipse |
|---|---|---|---|
| 3.54537328 | 4.67434614 | 2.89868951 | 5.7275816 |

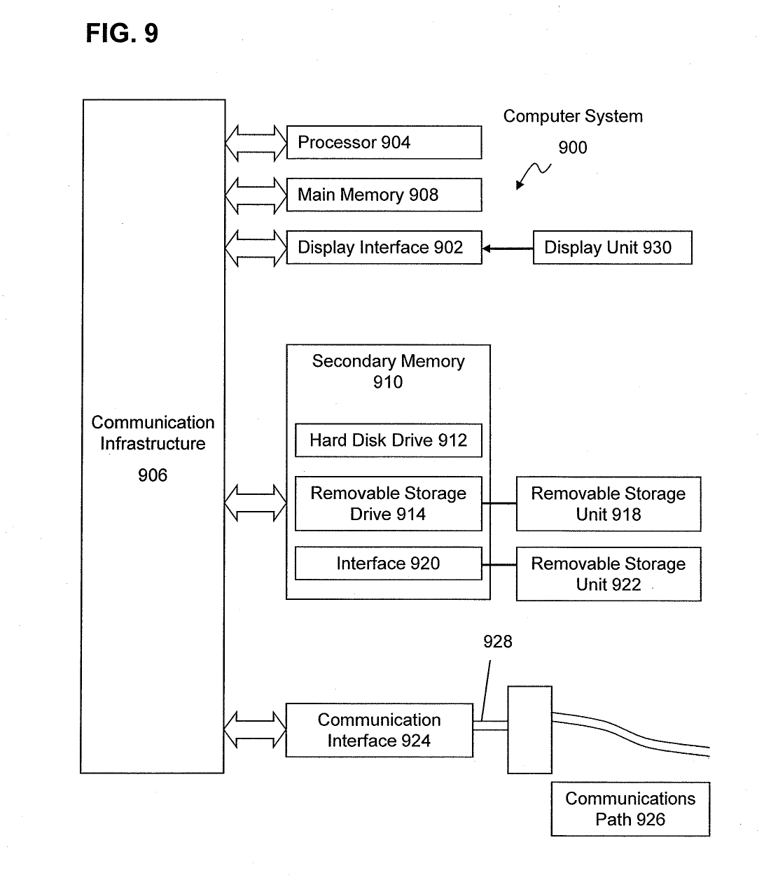

According to various aspects, the sensors within the platform may be monitored via a combination of hardware and software combination. For example, FIG. 9 presents an example system diagram of various hardware components and other features, for use in accordance with an aspect of the present invention. The present invention may be implemented using hardware, software, or a combination thereof and may be implemented in one or more computer systems or other processing systems. In one aspect, the invention is directed toward one or more computer systems capable of carrying out the functionality described herein. An example of such a computer system 900 is shown in FIG. 9.

Computer system 900 includes one or more processors, such as processor 904. The processor 904 is connected to a communication infrastructure 906 (e.g., a communications bus, cross-over bar, or network). Various software aspects are described in terms of this example computer system. After reading this description, it will become apparent to a person skilled in the relevant art(s) how to implement the invention using other computer systems and/or architectures.

Computer system 900 can include a display interface 902 that forwards graphics, text, and other data from the communication infrastructure 906 (or from a frame buffer not shown) for display on a display unit 930. Computer system 900 also includes a main memory 908, preferably random access memory (RAM), and may also include a secondary memory 910. The secondary memory 910 may include, for example, a hard disk drive 912 and/or a removable storage drive 914, representing a floppy disk drive, a magnetic tape drive, an optical disk drive, etc. The removable storage drive 914 reads from and/or writes to a removable storage unit 918 in a well-known manner. Removable storage unit 918, represents a floppy disk, magnetic tape, optical disk, etc., which is read by and written to removable storage drive 914. As will be appreciated, the removable storage unit 918 includes a computer usable storage medium having stored therein computer software and/or data.

In alternative aspects, secondary memory 910 may include other similar devices for allowing computer programs or other instructions to be loaded into computer system 900. Such devices may include, for example, a removable storage unit 922 and an interface 920. Examples of such may include a program cartridge and cartridge interface (such as that found in video game devices), a removable memory chip (such as an erasable programmable read only memory (EPROM), or programmable read only memory (PROM)) and associated socket, and other removable storage units 922 and interfaces 920, which allow software and data to be transferred from the removable storage unit 922 to computer system 900.

Computer system 900 may also include a communications interface 924. Communications interface 924 allows software and data to be transferred between computer system 900 and external devices. Examples of communications interface 924 may include a modem, a network interface (such as an Ethernet card), a communications port, a Personal Computer Memory Card International Association (PCMCIA) slot and card, etc. Software and data transferred via communications interface 924 are in the form of signals 928, which may be electronic, electromagnetic, optical or other signals capable of being received by communications interface 924. These signals 928 are provided to communications interface 924 via a communications path (e.g., channel) 926. This path 926 carries signals 928 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 980, a hard disk installed in hard disk drive 970, and signals 928. These computer program products provide software to the computer system 900. The invention is directed to such computer program products.

Computer programs (also referred to as computer control logic) are stored in main memory 908 and/or secondary memory 910. Computer programs may also be received via communications interface 924. Such computer programs, when executed, enable the computer system 900 to perform the features of the present invention, as discussed herein. In particular, the computer programs, when executed, enable the processor 910 to perform the features of the present invention. Accordingly, such computer programs represent controllers of the computer system 900.

In an aspect where the invention is implemented using software, the software may be stored in a computer program product and loaded into computer system 900 using removable storage drive 914, hard drive 912, or communications interface 920. The control logic (software), when executed by the processor 904, causes the processor 904 to perform the functions of the invention as described herein. In another aspect, the invention is implemented primarily in hardware using, for example, hardware components, such as application specific integrated circuits (ASICs). Implementation of the hardware state machine so as to perform the functions described herein will be apparent to persons skilled in the relevant art(s).

In yet another aspect, the invention is implemented using a combination of both hardware and software.

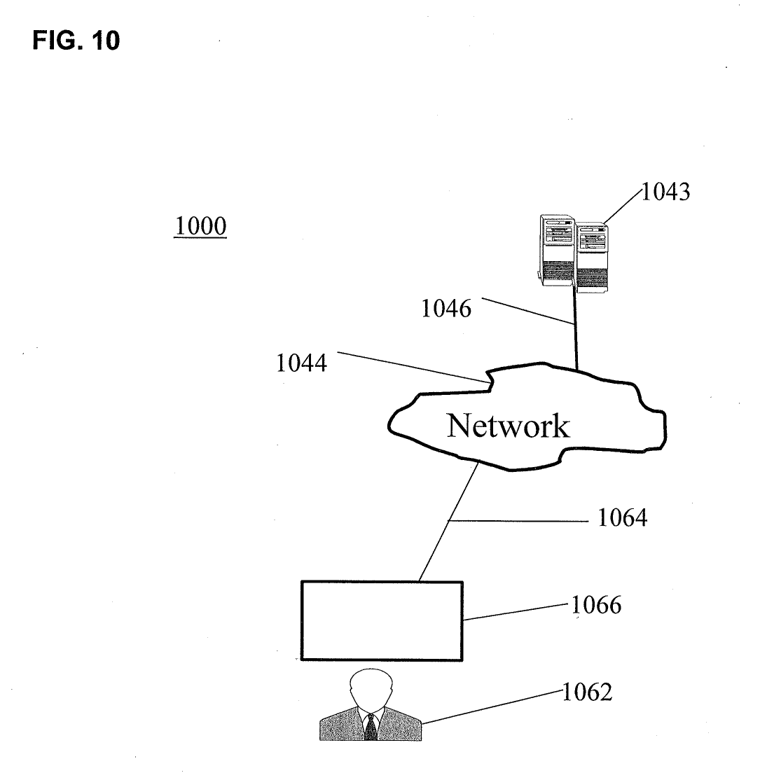

FIG. 10 is a block diagram of various example system components, in accordance with an aspect of the present invention. FIG. 10 shows a communication system 1000 usable in accordance with the present invention. The communication system 1000 may include one or more accessors 1062 (also referred to interchangeably herein as one or more “users”) and a terminal 1066. According to various aspects, the terminal 1066 may include a processor and one or more sensors such as the sensors described above and located in a device such as, e.g., a Wii balancing board as discussed above. In one aspect, data for use in accordance with the present invention is, for example, input and/or accessed by accessors 1062 via terminal 1066, such as a personal computer (PC), minicomputer, mainframe computer, microcomputer, telephonic device, or wireless devices, such as a personal digital assistant (“PDA”) or a hand-held wireless device, such device optionally further including, for example, one or more sensing devices and/or connections to such devices (e.g., a Wii balancing board), coupled to a server 1043, such as a PC, minicomputer, mainframe computer, microcomputer, or other device having a processor and a repository for data and/or connection to a repository for data, via, for example, a network 1044, such as the Internet or an intranet, and couplings 1046 and 1064. The couplings 1046 and 1064 include, for example, wired, wireless, or fiberoptic links.

Although the invention has been described with reference to various aspects and examples with respect to a system of posturography using a Wii balance board, it is within the scope and spirit of the invention to be incorporated in or used with any suitable system and/or mechanical device, and various alternatives, modifications, variations, improvements, and/or substantial equivalents, whether known or that are or may be presently unforeseen, may become apparent to those having at least ordinary skill in the art. Accordingly, the example aspects of the invention, as set forth above, are intended to be illustrative, not limiting. Therefore, it must be understood that numerous and varied modifications can be performed without departing from the spirit of the invention, and aspects of the invention are intended to embrace all known or later-developed alternatives, modifications, variations, improvements, and/or substantial equivalents.

1. A stabilometric system using a balance platform to detect an abnormality in the vestibular system of a person standing on the balance platform, comprising:
means for capturing data relative to weight distribution of the person on the balance platform over time via one or more sensors located at the balance platform;
means for displaying the captured data on a screen that is controlled by a computer; and
means for processing the captured data to determine the abnormality in the vestibular system.

2. The stabilometric system according to claim 1, wherein the balance platform comprises:
a Wii balance board that supports a maximum weight of 150 Kg,
a plurality of pressure sensors; and
a Bluetooth configured to communicate with the means for processing the captured data.

3. The stabilometric system according to claim 1, wherein the data displayed on the screen controlled by computer are represented in at least one of a statistical and a graphical way.

4. The stabilometric system according to claim 1, wherein the means for processing the captured data comprise means for determining the abnormality in the vestibular system based on the weight distribution of the person in the stability and posture of the person.

5. A method of detecting an abnormality in the vestibular system of a person standing on a balance platform, comprising:
capturing data relative to weight distribution of the person on the balance platform over time via one or more sensors located at the balance platform;
communicating the captured data to a processing device; and
processing the captured data to determine the abnormality in the vestibular system.

6. The method of claim 5, wherein the captured data is communicated to a display device for display.

7. The method of claim 6, wherein the data captured via the one or more sensors is communicated to the display device wirelessly.

8. The method of claim 5, wherein processing the captured data comprises determining the abnormality in the vestibular system of the person based on the weight distribution of the person over time on the balance platform.

9. The method of claim 6, wherein displaying the data comprises displaying the data in at least one of a statistical and a graphical way.

10. A stabilometric apparatus to detect an abnormality in the vestibular system of a person standing on a balance platform, comprising:
a plurality of sensors located at the balance platform, the plurality of sensors being located so as to detect a weight variation of the person over time on the balance platform and to generate an output; and
a data processing device to receive the output of the plurality of sensors via a communication device;
wherein the data processing device determines the abnormality in the vestibular system of the person based on the received output.

11. The apparatus of claim 10, wherein the plurality of sensors comprises one or more weight sensors.

12. The apparatus of claim 10, wherein the communication device is a wireless communication device.

13. The apparatus of claim 10, wherein the data processing device is configured to display the output of the plurality of sensors on a display device.
